(19) United States
(12) Plant Patent Application Publication
(10) Pub. No.: US 2016/0073563 P1
(43) Pub. Date: Mar. 10, 2016

(54) Westringia fruticosa plant named 'WES07'

(50) ;

(71) Applicant: Graham Brown, Cobbity (AU)

(72) Inventor: Graham Brown, Cobbity (AU)

(21) Appl. No.: 14/121,440

(22) Filed: Sep. 5, 2014

(51) Int. Cl. A01H 5/00

(52) U.S. Cl. PLT/226

(57) ABSTRACT

‘WES07’ is a distinctive variety of Westringia fruticosa which is characterized by the combination a small plant size, rotund shape, and a dense growth habit.

### LATIN NAME OF THE GENUS AND SPECIES

The Latin name of the genus and species of the novel variety disclosed herein is Westringia fruticosa.

### VARIETY DENOMINATION

The inventive variety of Westringia fruticosa disclosed herein has been given the variety denomination ‘WES07’.

### BACKGROUND OF THE INVENTION

The present invention relates to a new and distinct perennial variety of Westringia fruticosa, which has been given the variety denomination of ‘WES07’. Its market class is that of a shrub. ‘WES07’ is intended for use in general landscaping and as a decorative plant.

Parentage: The Westringia fruticosa variety ‘WES07’ is a seedling selection that resulted from the open pollination of male parent Westringia fruticosa (unpatented) and female parent line Westringia fruticosa ‘WES01’ (unpatented) at a commercial plant breeding facility in Cobbitty, NSW Australia. Seed from said cross was harvested in September of 2009 and was subsequently germinated in March of 2010 in accord with standard greenhouse propagation practices. The resulting young plants were later transplanted into 15 cm nursery containers and relocated to an outdoor trial area for field observations. Progeny were further observed for desired commercial characteristics and, in September 2011, one plant was selected for its rotund shape, compact size, and dense growth. In 2013, the cultivar was finally selected and named ‘WES07’.

Asexual Reproduction: ‘WES07’ was first propagated asexually by division in Cobbitty, New South Wales, Australia in September of 2011 and has since been asexually propagated by vegetative cuttings through 10 successive generations. The distinctive characteristics of the inventive ‘WES07’ variety are stable from generation to generation; clones of the variety produced by asexual reproduction maintain the distinguishing characteristics of the original plant.

### SUMMARY OF THE INVENTION

‘WES07’ is a distinctive variety of Westringia fruticosa which is characterized by the combination a small plant size, rotund shape, and a dense growth habit.

### BRIEF DESCRIPTION OF THE FIGURES

FIG. 1 shows an exemplary (approximate) 2-year old ‘WES07’ plant in the landscape.

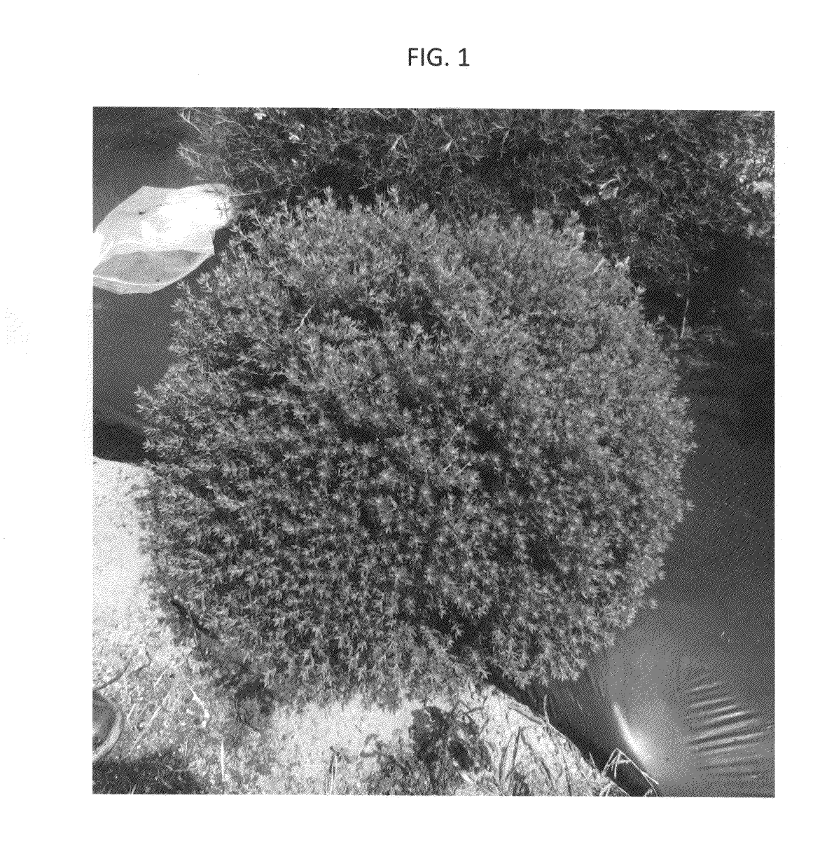

FIG. 2 shows an exemplary flower of ‘WES07’.

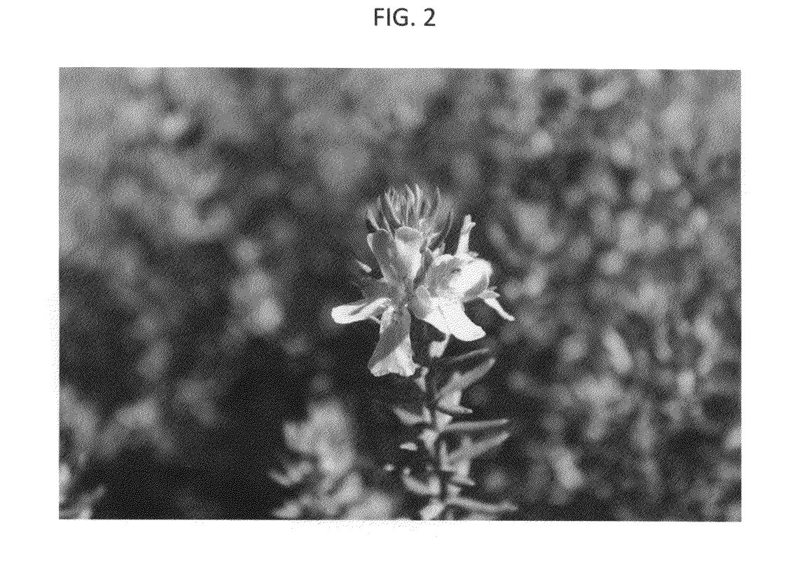

### BOTANICAL DESCRIPTION OF THE PLANT

The following is a detailed botanical description of a new and distinct variety of a Westringia fruticosa ornamental plant known as ‘WES07’. Unless indicated otherwise, the descriptions disclosed herein are based upon observations made from an (approximate) 2-year old ‘WES07’ plant, established in a full-sun landscape in Richmond, New South Wales, Australia. Said plant was transplanted into the landscape from a fully-rooted 15 cm nursery pot, grown in soilless potting media, from rooted cuttings from September 2011 to September 2013. Once transplanted into the landscape, the observed plant was maintained with granular slow release fertilizer and regularly watered with overhead irrigation. No pest and disease measures were taken.

Those skilled in the art will appreciate that certain characteristics will vary with older or, conversely, younger plants. ‘WES07’ has not been observed under all possible environmental conditions. Where dimensions, sizes, colors and other characteristics are given, it is to be understood that such characteristics are approximations or averages set forth as accurately as practicable. The phenotype of the variety may vary with variations in the environment such as season, temperature, light intensity, day length, cultural conditions and the like. Color notations are based on The Royal Horticultural Society Colour Chart, The Royal Horticultural Society, London, 2001 edition. Note that generic color descriptions such as ‘white’ do not exist in the R.H.S. charts and the corresponding R.H.S. colors are quoted.
Technical description of the variety.
Plant description:
   Plant habit.—Shrub; very short height and rotund in shape; with a dense growth habit.
   Height.—430 mm
   Width.—500 mm.
   Bloom period.—Spring and sporadically at other times of year, depending on climate.
   Hardiness.—USDA Zone 9 to 11.
   Propagation.—Propagation is accomplished using semi-hard wood cuttings. Roots well without rooting compound or hormone.
   Time to develop roots.—approximately 3 to 4 weeks.
   Crop time.—From approximately five to eight months are needed to produce a well rooted 15 cm nursery pot, starting from a rooted cutting. Crop time varies with location and production practices.
   Pest and disease susceptibility or resistance.—In common with the species, none of note.
Roots: The roots of ‘WES07’ are colored white and fleshy and thick, similar to other Westringia fruticosa.
Stem:
   Stem strength.—Medium rigidity.
   Attitude.—Prostrate to decumbent.
   Shape.—Cylindrical yet angular.
   Stem color.—Young stem is closest to green 138B.
   Stem dimensions.—mature stem, closest to plant's base, are approximately 350 mm in length with a diameter of 8 mm.
   Stem surface.—Corky and pruinose.
   Basal stem color.—Predominantly brown 200A with striations of both grey brown 199D and green 138A at maturity.
   Internode length.—Ranges from 8 mm in immature branches to 14 in mature branches.
Foliage:
   Type.—Evergreen.
   Shape.—Elliptical, narrow.
   Division.—Simple.
   Apex.—Acute.
   Base.—Cuneate.
   Venation.—reticulate but not obvious.
   Vein color (adaxial surfaces).—indistinguishable from surrounding foliage.
   Vein color(abaxial surfaces).—indistinguishable from surrounding foliage.
   Margins.—Entire.
   Arrangement.—Whorled.
   Attachment.—Petiolate.
   Texture.—Leathery.
   Surfaces (adaxial surface).—Finely pubescent to glabrous.
   Surfaces (abaxial surface).—Heavily pubescent.
   Mature leaf dimensions.—average length, 16 to 18 mm; width is 3 to 4 mm.
   Leaf color (adaxial surface).—Juvenile: 138B; mature: 139B.
   Leaf color (abaxial surface).—Juvenile: 138B; mature: 138B.
   Petiole.—Approximately 2 mm in length; width is approximately 1 mm.
   Stipules.—absent.
Inflorescence: Flowers are sessile.
Bud: Obovoid in shape and rounded at the apex. Approximately 5 mm long and 2.5 mm wide prior to dehiscence. Color is violet 85B.
Flower:
   General.—Flowers are sessile, simple and persist for approximately 5 days. Self cleaning. Non fragrant.
   Flowers.—Small; Sympetalous, irregular with petals numbering five, width across the corolla measuring on average 12 mm; length of the perianth, from base of calyx to the furthest outstretched point of the corolla, measures 14 mm; color approximates to purple 76A. Spots, or “eyes”, present; color closest to violet 84B. Margins cilliate; all surfaces are pubescent.
   Calyx.—Synsepalous with five pointed calyx lobes; color is green 137D. The length of the calyx, including sepal lobes, is 5 mm.
Reproductive organs:
   Stamens.—4 and 2 staminodes.
   Stamen color.—Purple 76A.
   Stamen dimensions.—About 4 mm in length and very thin.
   Attachment.—Adnate.
   Anther dimensions.—Bilobate; approximately 0.5 mm long.
   Anther color.—Violet 86D and slightly translucent.
   Pollen.—Absent.
   Pistil.—One.
   Pistil dimensions.—About 6 mm in length and very thin.
   Pistil color.—Purple 76A and slightly translucent.
   Ovary position.—Inferior.
Fruit and seed: Not observed.
Environmental tolerances:
Cold and heat tolerance: The winter hardiness of ‘WES07’ is at least to USDA Zone 9a and evaluation of winter hardiness is ongoing. ‘WES07’ has heat tolerance typical of Westringia fruticosa.
Drought tolerance: ‘WES07’ has good drought tolerance typical of Westringia fruticosa once established.
Pest resistance: No known insect pests. ‘WES07’ has disease tolerance typical of Westringia fruticosa.
Cultural conditions: Westringia is commonly known to perform well in sandy soils, but also tolerates heavy, clay-type soils well. ‘WES07’ can also be grown in a relatively wide pH range and is also tolerant of above-normal salt concentration in the soil environment and is also tolerant of aerosol salt exposure; e.g. coastal exposure to salt-laden winds.
   Comparison of WES07 with Other Varieties of Westringia fruticosa

‘WES07’ is similar to the seed parent, ‘NFL21’, in foliage shape, foliage color, and flower color. However, ‘WES07’ has a finer leaf texture, a much different plant growth habit and is significantly smaller in stature. The leaves of ‘WES07’ are smaller than those of ‘NFL21’, yet have a larger length to width ratio which gives them a finer texture. The leaves of ‘NFL21’ average 18.5 mm and 4.75 mm wide (length to width ratio value of 3.89) whereas the leaves of ‘WES07’ average 17.5 mm long and 3.5 mm wide (length to width ratio value of 5). The parent is a large variety (measuring 225 cm tall by 125 cm wide at maturity) with an upright and columnar growth habit whereas ‘WES07’ is a small variety (measuring 50 cm tall by 60 cm wide at maturity) with a “ball-shaped”, or rotund, growth habit.

There two additional Westringia fruticosa varieties selected by the breeder that share the same rotund growth habit exhibited by ‘WES07’. However, each is different in certain aspects.

The variety ‘WES02’ (U.S. Plant patent application No. 61/688,191) is a relatively small, rotund variety with green leaves and purple flowers, like ‘WES07’. However, ‘WES02’ has a coarser foliage texture and a larger plant size. ‘WES02’ has leaves that, on average, are 17 mm long and 5 mm wide (length to width ratio value of 3.4) whereas the leaves of ‘WES07’average 17.5 mm long and 3.5 mm wide (length to width ratio value of 5). ‘WES02’ measures 70 cm tall by 70 cm wide at maturity whereas ‘WES07’ measures 50 cm tall by 60 cm wide at maturity.

The second similar variety known to the breeder is ‘WES04’ which is also a small, rotund variety. While both ‘WES04’ and ‘WES07’ have a similar size and the same growth habit, there are significant differences in foliage and flower color. The leaves of ‘WES07’ are green (mature foliage corresponds to 139B) whereas the leaves of ‘WES04’ have more of a grey to greyed-green appearance due to their yellow-green upper leaf color (147A) and highly glaucous lower surface that creates a silvered appearance that corresponds closest to N155A. The flowers of ‘WES07’ are purple corresponding to purple 76A whereas the flowers of ‘WES04’ are white (no corresponding R.H.S. color but closest to 155D).

That which is claimed is:

1. A new and distinct variety of Westringia fruticosa plant named ‘WES07’, substantially as described and illustrated herein.
